(19) United States
(12) Patent Application Publication
(10) Pub. No.: US 2026/0198812 A1
(43) Pub. Date: Jul. 16, 2026

(54) FLEXIBLE DYNAMIC OPTICAL SPECTROSCOPIC SYSTEMS, DEVICES, AND METHODS FOR CARDIOVASCULAR MONITORING

(71) Applicant: New York University, New York, NY (US)

(72) Inventors: Albert Favate, Short Hills, NJ (US); Andreas Hielscher, Brooklyn, NY (US); Vadim Gordin, Brooklyn, NY (US); Allesandro Marone, Jersey City, NJ (US); Nisha Maheshwari, New York, NY (US); Lokesh Sharma, Brooklyn, NY (US); Stephen Kim, New York, NY (US)

(21) Appl. No.: 19/451,408

(22) Filed: Jan. 16, 2026

Related U.S. Application Data
(63) Continuation-in-part of application No. PCT/US2024/038690, filed on Jul. 19, 2024.
(60) Provisional application No. 63/514,449, filed on Jul. 19, 2023.

(51) Int. Cl. A61B 5/1455; A61B 5/00; A61B 5/145

(52) U.S. Cl. CPC A61B 5/14552; A61B 5/14539; A61B 5/14546; A61B 5/6822; A61B 5/6833; A61B 2562/0238; A61B 2562/046; A61B 2562/164

(57) ABSTRACT

An optical spectroscopic device for cardiovascular monitoring, comprising a housing, an attachment mechanism for attaching the housing proximate to a target tissue location, a plurality of light sources positioned on the housing, each configured to emit light at one of a plurality of wavelengths, a plurality of photodetectors proximate to the light sources and positioned on the housing, configured to measure the light emitted by the light sources that transmits through or reflects off the target tissue location, a transducer configured to convert voltage levels measured at the plurality of photodetectors to digital measurement data, and a communications module electrically connected to the transducer, configured to transmit the digital measurement data to a computing system configured to calculate a property of the blood, plaque, and/or carotid artery within the target tissue location based on the digital measurement data.

### CROSS-REFERENCE TO RELATED APPLICATIONS

This application is a Continuation-In-Part of and claims benefit to International Patent Application No. PCT/US24/38690 filed on Jul. 19, 2024, which is entitled to priority of U.S. provisional application No. 63/514,449 filed on Jul. 19, 2023, each of which are incorporated herein by reference in their entirety.

### BACKGROUND OF THE INVENTION

Stroke is a leading cause of long-term disability globally, and outcomes worsen the longer a patient waits to get treated. The carotid body is a small cluster of chemoreceptors and supporting cells located near the bifurcation of the carotid artery. Its primary function is to sense changes in the composition of arterial blood, specifically the concentrations of oxygen, carbon dioxide, and the pH level. The carotid body is a part of the peripheral chemoreceptors and is the body's primary oxygen sensor. During hypoxia (decrease in oxygen levels in the blood), hypercapnia (increase in carbon dioxide levels in the blood), or acidosis (when blood becomes more acidic) event, the carotid body sends signals to the respiratory center in the brainstem to adjust the rate and depth of respiration and return the chromophores and pH of the blood to their normal levels.

The carotid body is highly vascularized, receiving its blood supply from the carotid artery, which enables it to monitor arterial blood directly. It contains two types of cells: Glomus cells and Sustentacular cells. The Glomus cells (Type I cells) detect changes in blood oxygen, carbon dioxide, and pH levels. Upon stimulation, these cells release neurotransmitters, including dopamine and acetylcholine, which initiate nerve impulses in the sensory fibers of the carotid sinus nerve, a branch of the glossopharyngeal nerve. The Sustentacular cells (Type II cells) are supporting cells thought to play a role in the structural organization of the carotid body and possibly in regulating the function of the Glomus cells.

The ability to monitor the carotid arteries and stimulate the carotid body may lead to better treatment and prevention of various diseases and medical conditions. Thus there is a need in the art for improved monitoring and stimulation devices, systems, and methods.

### SUMMARY OF THE INVENTION

Some embodiments of the invention disclosed herein are set forth below, and any combination of these embodiments (or portions thereof) may be made to define another embodiment.

In one aspect, an optical spectroscopic device for cardiovascular monitoring comprises a housing, an attachment mechanism for attaching the housing proximate to a target tissue location, a plurality of light sources positioned on the housing, each configured to emit light at one of a plurality of wavelengths, a plurality of photodetectors proximate to the light sources and positioned on the housing, configured to measure the light emitted by the light sources that transmits through or reflects off of structures in the target tissue location, a transducer configured to convert voltage levels measured at the plurality of photodetectors to digital measurement data, and a communications module electrically connected to a transducer, configured to transmit the digital measurement data to a computing system configured to calculate a property of the blood and/or plaque within the target tissue location based on the digital measurement data.

In one embodiment, the light sources comprise light-emitting diodes.

In another embodiment, the light sources are laser diodes.

In one embodiment, the photodetectors comprise a plurality of silicon photodiodes and Indium Gallium Arsenide (InGaAs) detectors.

In one embodiment, the light sources are positioned in a columnar configuration.

In one embodiment, the attachment mechanism comprises a biocompatible adhesive or a strap.

In one embodiment, each of the photodetectors is positioned 10 mm to 70 mm from at least one of the light sources.

In one embodiment, the housing is flexible.

In one embodiment, the plurality of light sources comprises 16 light sources and the plurality of photodetectors comprises 12 photodetectors.

In one embodiment, the device is configured for placement proximate to a carotid artery.

In one embodiment, all of the wavelengths emitted by the light sources are in the range of 650 nm to 1500 nm.

In one embodiment, the plurality of light sources are positioned in first and second groups, with the first of the plurality of photodetectors positioned between the first and second groups of light sources, with a second of the plurality of the photodetectors positioned on a side of the first group of light sources opposite the first photodetectors, with a third of the of the plurality of the photodetectors positioned on a side of the second group of light sources opposite the first photodetectors.

In one embodiment, the device is configured to remotely monitor for strokes.

In one embodiment, the device is configured for outpatient monitoring of the carotid artery.

In one embodiment, the device is configured to determine blood pH.

In one embodiment, the device is non-invasive.

In one embodiment, the device is configured to measure plaque composition.

In one embodiment, the device is configured to track the movement of plaque through the carotid artery.

In one embodiment, the device is configured to provide 3D images of the vasculature in the neck.

In another aspect, an optical spectroscopic system for cardiovascular monitoring comprises the optical spectroscopic device as described above, and a computing system communicatively connected to the optical spectroscopic device, comprising a processor and a non-transitory computer-readable medium with instructions stored thereon, which when executed by the processor, perform steps comprising applying light of several wavelengths to the target tissue location, receiving the digital measurement data transmitted by the optical spectroscopic device, and calculating a property of the blood and/or plaque within the target tissue location based on the digital measurement data.

In another aspect, a cardiovascular monitoring method comprises providing the optical spectroscopic device or system as described above, applying a plurality of wavelengths of light to the target tissue location with the plurality of light sources, measuring transmission or reflection of light at the target tissue location with the plurality of photodetectors, and calculating a property of the blood and/or plaque within the target tissue location.

In one embodiment, the method further comprises applying a stimulation comprising electrical impulses via an electrode positioned on the housing of the device to stimulate cerebral blood profusion.

In one embodiment, the method further comprises applying a stimulation comprising ultrasound waves via an ultrasound transducer positioned on the housing of the device to stimulate the carotid body.

In one embodiment, the calculated property comprises at least one of blood pH, plaque composition, plaque location, blood oxygenation, and blood concentration.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 depicts an exemplary optical spectroscopic device for cardiovascular monitoring in accordance with some embodiments.

FIG. 2 depicts an exemplary housing of the device in FIG. 1 in accordance with some embodiments.

FIG. 3 depicts an exemplary schematic layout of light sources and photodetectors of the device of FIG. 1 in accordance with some embodiments.

FIG. 4 depicts another exemplary optical spectroscopic device for cardiovascular monitoring in accordance with some embodiments.

FIG. 5 depicts an exemplary housing of the device of FIG. 4 in accordance with some embodiments.

FIG. 6 depicts an exemplary schematic layout of light sources and photodetectors of the device of FIG. 4 in accordance with some embodiments.

FIG. 7 depicts another exemplary optical spectroscopic device for cardiovascular monitoring in accordance with some embodiments.

FIG. 8 depicts a cardiovascular monitoring method in accordance with some embodiments.

FIGS. 9-11 depict exemplary experimental results, using the device of FIG. 4, of axial cross-section image reconstructions of the neck vasculature in three healthy subjects at peak of breath hold based on oxyhemoglobin and deoxyhemoglobin concentration reconstructions in accordance with some embodiments.

FIG. 12 depicts exemplary experimental results, using the device of FIG. 4, of axial cross-section reconstructions in one healthy subject (same subject as FIG. 10) during normal breathing and at the peak of breath hold based on dynamic changes in total hemoglobin concentration data in accordance with some embodiments.

FIG. 13A depicts a coronal cross-section of the left carotid artery during a breath hold of a fourth healthy subject, imaged with carotid Doppler ultrasound.

FIG. 13B depicts an axial cross-section of the left carotid artery during a breath hold of the subject in FIG. 13A, imaged using the device of FIG. 4 and reconstructed with a third-order spherical harmonics algorithm.

FIG. 14A shows a coronal cross-section of the right carotid artery during a breath hold of the same subject associated with FIGS. 10 and 12, imaged with carotid Doppler ultrasound.

FIG. 14B shows an axial cross-section of the right carotid artery during a breath hold of the subject in FIG. 14A, imaged using the device of FIG. 4 and reconstructed with a third-order spherical harmonics algorithm.

FIG. 15 depicts a simulation of plaque within the right carotid artery of a neck in accordance with some embodiments.

FIG. 16A depicts coronal cross-sections of the simulated distribution of cholesterol (left) and oxyhemoglobin (right) in the simulated neck characterized by FIG. 15.

FIG. 16B depicts coronal cross-sections of the cholesterol (left) and oxyhemoglobin (right) detection simulation results showing the exemplary configuration of FIG. 3 resolving the height and location of the plaque characterized by FIG. 15.

FIG. 17 shows an exemplary acquisition screen of a MATLAB GUI, where users can specify the side of the neck and number of devices in use, and start acquiring data.

FIG. 18 depicts an exemplary computing environment in which aspects of the invention may be practiced.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in flexible dynamic optical spectroscopic systems, devices, and methods for cardiovascular monitoring. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein are flexible dynamic optical spectroscopic systems, devices, and methods for cardiovascular monitoring.

The carotid body, being a vital component of the body's chemoreceptor system, can be associated with several health problems. These include:
Sleep Apnea: Dysfunction of the carotid body has been implicated in sleep apnea; a condition characterized by repeated pauses in breathing during sleep. The carotid body's abnormal response to oxygen levels can contribute to disruptions in the respiratory control system.
Hypertension (High Blood Pressure): The carotid body plays a role in regulating blood pressure. Dysregulation of the carotid body's chemosensing function may contribute to excessive sympathetic nervous system activation, leading to hypertension.
Heart Failure: In heart failure, the carotid body can become hyperactive, leading to sympathetic overactivity and further deterioration of heart function. The abnormal signaling from the carotid body may exacerbate heart failure symptoms.
Metabolic Disorders: Emerging evidence suggests a potential link between the carotid body and metabolic disorders such as insulin resistance and obesity. Dysfunctional carotid body responses may influence metabolic regulation and contribute to the development or progression of these conditions.
Chronic Mountain Sickness (CMS): At high altitudes, low oxygen levels can trigger excessive carotid body activity, resulting in symptoms of CMS. These may include headache, fatigue, shortness of breath, and sleep disturbances.

Despite many health conditions associated with the carotid bodies, and the critical job of the carotid arteries as the primary blood supply to the brain, there have been no non-invasive medical devices that can monitor and stimulate the carotid body and artery to activate the baroreflex.

Hence, a novel approach is needed for a next-gen medical device that can non-invasively sense and/or activate baroreflex among individuals by monitoring and/or stimulating the carotid bodies and arteries.

In some embodiments, an optical spectroscopic device 100 for cardiovascular monitoring comprises a housing 101, and an attachment mechanism 102 such as a strap or biocompatible adhesive for attaching the housing 101 proximate to a target tissue location. The target tissue location can include the carotid artery, carotid body, radial artery, or other suitable location. In some embodiments, the device 100 further includes a plurality of light sources 103 positioned on the housing 101, with each configured to emit light at one of a plurality of wavelengths. In some embodiments, the device 100 further comprises a plurality of photodetectors 104 proximate to the light sources 103 and positioned on the housing 101, each configured to measure the light emitted by the light sources 103 that transmits through or reflects off the target tissue location. In some exemplary embodiments, a communications module 105 resides in the housing 101. The communications module 105 is electrically connected to the light sources 103 and photodetectors 104 and configured to transmit the digital measurement data to a computing system 198 configured to calculate a property of the blood, plaque, and/or artery within the target tissue location based on the digital measurement data. In some embodiments, the device 100 can include a transducer 106 configured to convert voltage levels measured at the plurality of photodetectors 104 to digital measurement data, and a communications module 105 external to the housing 101 and electrically connected to the light sources 103, photodetectors 104, and/or transducer 106, configured to transmit the digital measurement data to a computing system 198 configured to calculate a property of the blood, plaque, and/or artery within the target tissue location based on the digital measurement data. In some embodiments, the device 100 is a multi-use device.

In some embodiments, an optical spectroscopic system 199 for cardiovascular monitoring comprises the optical spectroscopic device 100, and a computing system 198 communicatively connected to the optical spectroscopic device, comprising a processor and a non-transitory computer-readable medium with instructions stored thereon, which when executed by the processor, perform steps comprising applying light of several wavelengths to the target tissue location, receiving the digital measurement data transmitted by the optical spectroscopic device 100, and calculating a property of the blood, plaque, and/or artery within the target tissue location based on the digital measurement data. The optical spectroscopic device 100 may be communicatively connected to the computing system 198 by any suitable wired or wireless data connection, including but not limited to SPI, I2C, Bluetooth, Bluetooth LE, and/or Wi-Fi.

In some embodiments the light sources 103 comprise laser diodes, light emitting diodes, or other suitable light sources. In some embodiments, at least a portion of the light sources 103 are positioned in a square configuration. In some embodiments, all of the multiple wavelengths emitted by the light sources are in the range of 650 nm to 1500 nm. In some embodiments, each light source may emit light in principally a single wavelength, i.e. having a roughly bell-curve shaped emission spectrum centered around a single center wavelength, and in other embodiments, one or more of the light sources may individually emit light in multiple wavelengths, i.e. have a more complex emission spectrum with multiple local maxima. Center emission wavelengths of an exemplary embodiment include but are not limited to wavelengths of 670 nm, 730 nm, 760 nm, 780 nm, 850 nm, 870 nm, 1200 nm, 1450 nm, and 1460 nm.

In some embodiments, the photodetectors comprise silicon (Si) photodiodes, indium gallium arsenide (InGaAs) photodiodes, dual band (Si/InGaAs) photodiodes, germanium (Ge) photodiodes, or other suitable photodetectors. In some embodiments, each of the photodetectors 104 is positioned 10 mm to 70 mm from at least one of the light sources.

In some embodiments, the housing 101 is flexible. In some embodiments, the housing is 3D printed. In some embodiments, the housing comprises plastic, rubber, silicone, polylactic acid (PLA), acrylonitrile butadiene styrene (ABS), thermoplastic elastomers (TPE) including but not limited to thermoplastic polyurethane (TPU), polypropylene (PP), thermoplastic copolyester (TPC), ethylene propylene diene monomer (EPDM), or similar flexible material. In some embodiments, the housing material is biocompatible. In some embodiments, the device 100 is placed proximate to the target tissue location such that photodetector 104 detection surfaces are at distances in the range of 20 mm to 115 mm from the emission point of each light source.

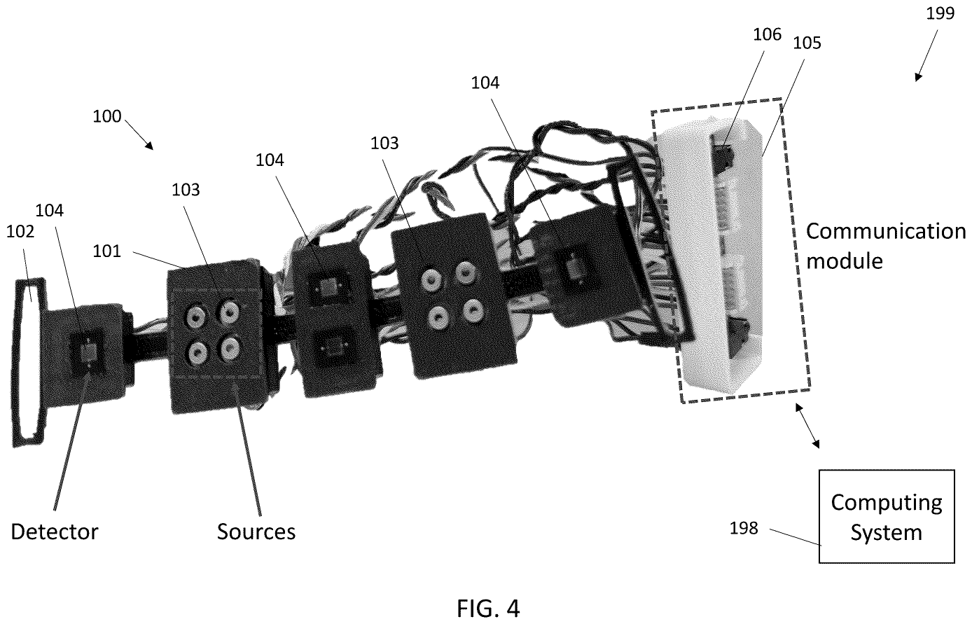

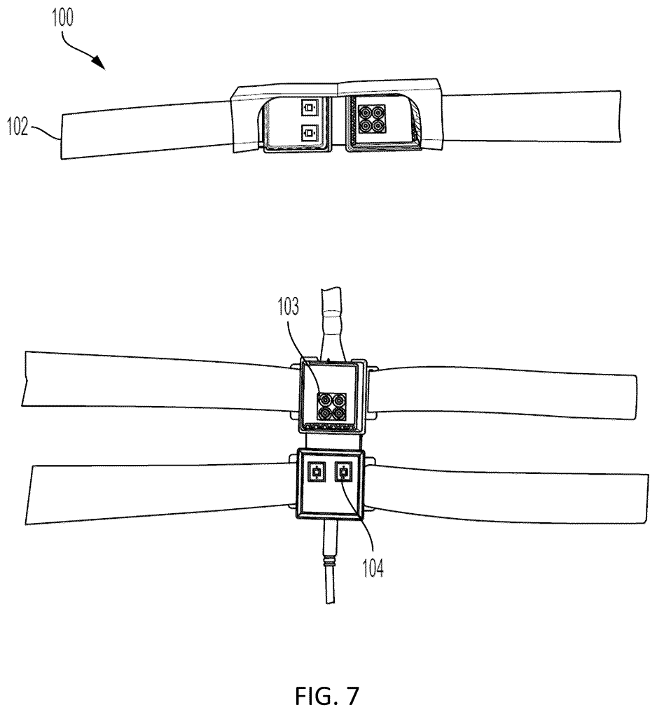

In some embodiments, the plurality of light sources 103 comprises sixteen light sources and the plurality of photodetectors 104 comprises twelve photodetectors. In some embodiments, the plurality of light sources 103 comprises eight light sources and the plurality of photodetectors 104 comprises four photodetectors. In some embodiments, the plurality of light sources 103 are positioned in first and second groups. In one embodiment, a first group of four of the plurality of photodetectors 104 are positioned between the first and second groups of light sources 103, with a second group of four of the plurality of photodetectors 104 positioned on a side of the first group of light sources 103 opposite the first group of photodetectors, with third group of four of the plurality of photodetectors 104 on a side of the second group of light sources 103 opposite the first and second groups of photodetectors. (FIG. 4) In another embodiment, a first and a second of the plurality of photodetectors 104 positioned between the first and second groups of light sources 103, with a third of the plurality of the photodetectors 104 positioned on a side of the first group of light sources 103 opposite the first and second photodetectors 104, with a fourth of the of the plurality of the photodetectors 104 positioned on a side of the second group of light sources 103 opposite the first and second photodetectors 104. (FIG. 7)

In some embodiments, the device 100 is configured to remotely monitor for strokes. In some embodiments, the device 100 is configured for outpatient monitoring of the carotid artery. In some embodiments, the device 100 is configured to remotely monitor embolic activity through the carotid artery. In some embodiments, the device 100 is configured for outpatient monitoring of microemboli. In some embodiments, the device 100 is configured to determine blood pH. In some embodiments, the device 100 is non-invasive. In some embodiments, the device 100 is configured to measure plaque composition.

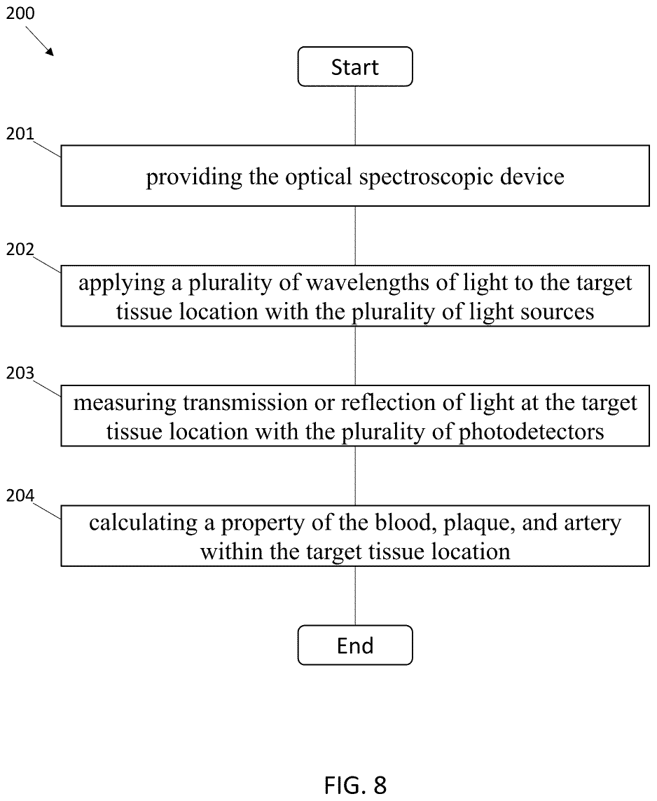

In some embodiments, a cardiovascular monitoring method 200 (FIG. 8) comprises a step 201 comprising providing the optical spectroscopic device 100 and or system 199, a step 202 comprising applying a plurality of wavelengths of light to the target tissue location with the plurality of light sources 103, a step 203 comprising measuring transmission or reflection of light at the target tissue location with the plurality of photodetectors 104, and a step 204 comprising calculating a property of the blood, plaque, and/or artery within the target tissue location. In some embodiments, the calculated property comprises at least one of blood pH, plaque composition, plaque location, blood concentration, and/or blood oxygenation.

In some embodiments, the method 200 further includes an optional step comprising applying a stimulation comprising electrical impulses via an electrode positioned on the housing of the device to stimulate cerebral blood profusion. In some embodiments, the method 200 further includes an optional step comprising applying a stimulation comprising ultrasound waves via an ultrasound transducer positioned on the housing of the device to stimulate the carotid body.

### Experimental Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore specifically point out exemplary embodiments of the present invention and are not to be construed as limiting in any way the remainder of the disclosure.

Stroke is a leading cause of long-term disability globally, and outcomes worsen the longer a patient waits to get treated. Despite this, there are no commercially available outpatient monitoring devices for stroke. A non-invasive, wearable patch was developed to address this gap in patient care (FIG. 7). The device uses dynamic vascular optical spectroscopy (DVOS) to record real-time changes in the blood flow through the carotid arteries. The flexible patch houses four sources of wavelengths 670 nm, 780 nm, 808 nm, and 850 nm and two silicon photodetectors. The patch has a 16.5 mm depth resolution and continuously records reflected light intensity in the carotid artery and associated tissue at 2.24 Hz. Below are details of a preclinical study in which the left and right radial and carotid arteries of 5 healthy subjects were monitored simultaneously using four DVOS patches. The location and size of the left and right carotid arteries were also determined for each subject via carotid Doppler ultrasound. Subjects were asked to perform a 30 second breath hold, after which the DVOS-derived changes in oxy-, deoxy-, and total hemoglobin concentrations of all four arteries were analyzed. Preliminary results show no statistically significant differences between the radial and carotid arteries for these three parameters. Healthy subjects do not have plaque in their carotid arteries or other stroke risk factors, thus it is expected that their radial and carotid arteries will have the same relative changes in blood concentration, as is reflected in these results. The DVOS-derived baseline total hemoglobin concentration in the left carotid was highly correlated with the left carotid bulb diameter (R=0.6) and the left internal carotid artery diameter (R=0.7). The DVOS-derived maximum change in oxyhemoglobin concentration in the right carotid was highly correlated with the right internal carotid artery diameter (R=0.6).

Tracking hemodynamic changes in the carotid arteries of patients at high risk for stroke may help these patients get timely treatment in response to stroke symptoms. A wearable, non-invasive patch has been developed which uses dynamic vascular optical spectroscopy to track real-time changes in cerebral blood perfusion through the carotid arteries. The changes in blood flow were compared between the carotid and radial arteries of 5 healthy subjects in response to a 30 second breath hold. No differences in the change of oxy- and deoxyhemoglobin were found between the radial and carotid arteries, as expected from healthy subjects.

Further, a flexible 3-D printed patch for dynamic optical spectroscopy has been developed to evaluate the total blood oxygenation within the carotid artery. Carotid artery monitoring is crucial for the early detection of abnormalities and offers insight into personalized healthcare. Each experimental patch comprised of a sensing module and detection module measuring 25 mm×20 mm and 21 mm×20 mm, respectively, separated by a distance of approximately 25 mm and 32 mm. The sensing module comprised of four sources at wavelengths of 670 nm, 750 nm, 808 nm, and 850 nm placed in a square configuration, and the detection module included two photodiodes in a parallel orientation (FIG. 7). During the testing phase, two probes (FIG. 7 top panel) were applied to the neck, proximally to both the left and right carotid arteries (with carotid artery location determined via carotid Doppler ultrasound), and two probes (FIG. 7 bottom panel) were also placed on the right and left wrists, proximally to the radial arteries (which were located via visual inspection). Six healthy participants were instructed to perform breathing exercises, such as a single deep breath, continuous deep breaths, and a timed breath hold, while allowing for intervals of routine breathing between each activity. Analysis of these time intervals enabled identifying the increasing, decreasing, and regular blood oxygenation levels. The study demonstrated consistent blood oxygen content between the left and right carotid and radial arteries. Additionally, during a breath hold, a 0.3% and a 0.1% decrease was observed in oxygen saturation in the radial and carotid arteries, respectively. These findings underscore the patch system's potential in detecting disease-related variations in individual carotid arteries, facilitating early detection.

The developed device, called CaroSense, is non-invasive, eliminating the need for surgical procedures and reducing associated risks and complications. This ensures a safer and more patient-friendly experience.

CaroSense can implement advanced monitoring capabilities to track and analyze baroreflex response in real-time. This feedback can enable healthcare providers to make informed adjustments and optimize treatment protocols for each patient, as well as help patients understand their vascular health and make decisions on when to seek care.

CaroSense has the ability to be an intuitive and user-friendly medical device that allows patients to easily monitor their therapy. Using real-time feedback, physicians can ensure patient engagement in managing their condition and develop more effective treatments.

CaroSense is a medical device that can non-invasively monitor cerebral blood perfusion. This system is based on the natural ability of the carotid body to control the blood flow to the brain, using a hemostatic mechanism that helps maintain blood pressure at nearly constant levels. Hence, the current invention discloses methods and devices for the non-invasive monitoring of the carotid body and related structures, such as the internal and external carotid arteries. Such techniques can potentially provide benefits for managing various health conditions, including heart failure, sleep apnea, chronic obstructive pulmonary disease (COPD), and metabolic disorders such as insulin resistance, obesity, and diabetes, through early detection.

### Carosense

The first prototype (FIG. 7) uses dynamic vascular optical spectroscopy (DVOS) to continuously measure the oxygen saturation of and blood flow through the carotid artery. Monitoring oxygen saturation and changes in total hemoglobin concentration can provide information on the vascular health of an artery and alert to potential health risks, such as transient ischemic attack (TIA) or stroke, which are affected by the delivery of oxygen to the brain.

DVOS is a non-invasive, non-ionizing, and contrast-free technique that uses red and near-infrared light to penetrate tissues of interest and provide information on chromophores in those tissues. Previous research has shown that DVOS and similar techniques can distinguish between healthy subjects and patients with peripheral arterial disease (PAD), a vascular disease, and can classify wound healing outcomes for PAD patients after surgical intervention. These findings show that the hemodynamic changes observed in the target tissue in response to external stimuli differ based on the relative health of the vasculature. The DVOS measurements were used to find the total hemoglobin concentration over time at various arteries of interest. Variables extracted from the total hemoglobin concentration show high sensitivity and specificity in monitoring vascular health in the lower extremities.

Unlike current technologies, such as Doppler ultrasound, the disclosed DVOS system provides real-time information on changes in oxygenated and deoxygenated hemoglobin concentrations (i.e., changes in oxygen saturation). The system has up to four probes that can collect information on oxygen saturation and is calibrated based on the oxygen saturation in the left and right radial arteries, which have the same oxygen saturation as the carotid arteries for a healthy subject. One can monitor changes in both the left and right carotid arteries simultaneously and determine the localized area(s) of risk.

### Design and Hardware

The first CaroSense prototype is a DVOS system that analyzes the absorption and reflection of light within the red and near-infrared spectrum to determine hemoglobin concentration in an area of interest. The developed patch for carotid oxygenation monitoring comprises a separate laser-diode array module and light-detection module measuring 25 mm×20 mm and 21 mm×20 mm, respectively. The laser-diode array module contains four sources at wavelengths of 670 nm, 750 nm, 808 nm, and 850 nm (sourced from Thorlabs: HL6748MG, L780P010, L808P010, L850P010) placed in a square configuration (FIG. 7). Each laser diode operates at 10 mW power and is modulated at a frequency of 5 KHz. The light-detection module contains two silicon photodiodes (Hamamatsu S1337-33BR) placed in a parallel orientation. The sources and detectors are separated by a distance of approximately 25 mm and 32 mm for the two columns of sources, respectively, and contained within a flexible 3-D printed housing of 85 mm×30 mm (FIG. 7). The top panel shows a patch for monitoring the carotid artery with two photodetectors (left) and four laser diodes (right) in a flexible 3D housing. The bottom panel shows a patch for monitoring the radial artery with cables used for acquiring data. A Velcro strap attached to the 3D housing conforms to fit around the subject's neck or wrist and adheres the patch to the skin during monitoring. Each end of the 3-D printed housing has an opening for the cables that connect the data acquisition hardware and the source and detector modules (FIG. 7).

### Additional Implementations with Carosense

Monitoring the composition of arterial plaques is implemented using a broader spectrum of light to identify more chromophores of interest. Using modern approaches, CaroSense can provide clinicians with valuable real-time information about plaque characteristics. This enhanced monitoring capability holds the potential to further refine treatment decisions, risk assessment, and overall patient management. Expanding the functionalities of CaroSense improves patient outcomes through precise and comprehensive monitoring of arterial plaques. We propose the following techniques to achieve this goal:

To implement a transcutaneous emitter-detector pair a light source and detector are placed on the skin surface above the target artery. The source emits light of a broad spectrum, which penetrates the skin and interacts with the arterial plaque. The detector measures the reflected or transmitted light, capturing valuable information about the plaque's composition. By analyzing the spectral properties of the reflected/transmitted light, characteristics such as plaque composition, lipid content, and collagen deposition can be assessed.

To implement a laparoscopy-style probe or stylet involves inserting a probe or stylet with an integrated light source and detector under the skin through minimally invasive laparoscopic or endoscopic techniques, without puncturing the artery. The probe is placed over the artery and the emitted light interacts with the plaque, while the detector measures the spectral changes in the reflected or transmitted light, providing information about plaque composition. This method enables a more localized and precise assessment of the arterial plaque's characteristics.

Both approaches utilizing broader spectrum light offer non-destructive and real-time evaluation of arterial plaques. They have the potential to provide clinicians with valuable insights into plaque composition, helping guide treatment decisions and risk stratification. They also have the potential to inform patients on when it is time to seek care for carotid stenosis, stroke, or other related cardiovascular diseases.

### Caroactivate

To activate the baroreceptor signaling pathway, the system has to transmit the impulses via the glossopharyngeal of the carotid body to modulate sympathetic and parasympathetic neurons in the medulla/pontine area. This can be achieved with excitatory postsynaptic potential (EPSP) generation within the carotid body to influence the Nucleus Tractus Solitarii (NTS) of the brainstem.

Non-invasive vagus nerve stimulation (nVNS) is a method used to stimulate the vagus nerve without requiring surgical implantation of a device. This therapy typically involves a handheld device that delivers electrical impulses to the vagus nerve through the skin. The vagus nerve is the tenth cranial nerve and a major component of the parasympathetic nervous system, which controls functions such as heart rate and digestion. It also has afferent (sensory) fibers that communicate sensory information from the periphery to the brain, including the sensory information from baroreceptors and chemoreceptors in the cardiovascular system. In general, nVNS is applied on the neck, where the vagus nerve runs close to the skin surface, usually over the carotid artery region. The device delivers a mild electrical signal through the skin to stimulate the nerve.

The carotid body, a small, highly vascularized structure located at the bifurcation of the carotid artery, functions as a chemoreceptor, detecting changes in blood oxygen and carbon dioxide levels. It primarily communicates with the brain through the glossopharyngeal nerve (ninth cranial nerve), sending signals that influence respiratory and cardiovascular functions. Similar to the carotid sinus, the vagus nerve may have a role in modulating the activity of the carotid body. Hence, nVNS might influence the chemoreceptor activity in the carotid body, affecting the control of respiration. The potential of nVNS to stimulate the carotid body lies in the interconnectedness of the nervous system. Although the vagus nerve (tenth cranial nerve) and glossopharyngeal nerve are separate, they have close anatomical relationships and possible functional crosstalk. During nVNS, a handheld device is applied to the neck, where the vagus nerve runs close to the skin. By delivering a mild electrical signal, nVNS might indirectly influence the carotid body. The stimulation could modulate the activity of the carotid body and thereby influence respiratory control, potentially providing a therapeutic avenue for conditions like sleep apnea or certain metabolic disorders.

High-Intensity Focused Ultrasound (HiFU) is a non-invasive therapeutic technique that uses non-ionizing ultrasonic waves to heat tissue. It has been successfully applied in treating a range of solid malignancies, including those of the liver, prostate, breast, pancreas, bone, and uterine fibroids. The principle of HiFU involves focusing sound waves on creating a point of high energy within the body; this energy causes a rapid temperature rise at the focus point, leading to localized cell death and tissue destruction. In theory, HiFU could be used to target and modulate the activity of the carotid body. This approach would involve using ultrasound waves to heat specific regions of the carotid body, causing controlled cellular changes that alter its activity. By carefully controlling the intensity and focus of the ultrasound waves, it might be possible to modulate the carotid body's chemosensing function, potentially providing a therapeutic avenue for conditions like sleep apnea or certain metabolic disorders.

An implantable bioelectronic stimulation device or optogenetic stimulation device can be developed as an alternative to non-invasive techniques for carotid stimulation. The bioelectronic device could be designed as a cuff around the bifurcation of the carotid artery to deliver precise electrical signals to the desired area. Similarly, an optogenetic stimulation cuff could use light to control cells in living tissue, typically neurons, which have been genetically modified to express light-sensitive ion channels. When such channels are exposed to light (often through a fiber-optic cable), they open or close, thus triggering or inhibiting neuronal activity. There have been certain studies focusing on photobiomodulation and stroke recovery using optogenetics.

At present, the first iteration of CaroSense has been developed using dynamic vascular optical spectroscopy (DVOS) to continuously measure the oxygen saturation of the carotid artery. The designed DVOS system has been previously employed for a different vascular health condition (peripheral arterial disease) and has undergone clinical pilot studies that validated the use of DVOS in differentiating between relative vascular healthiness of tissue regions in the legs and feet and in monitoring wound healing in patients with a lower extremity vascular disease. This system was also able to continuously acquire data for 1-3 hours. (see M. A. Khalil et al., ‘Detection of Peripheral Arterial Disease Within the Foot Using Vascular Optical Tomographic Imaging: A Clinical Pilot Study’, 2015)(see N. Maheshwari et al. ‘Postintervention monitoring of peripheral arterial disease wound healing using dynamic vascular optical spectroscopy’, 2022) (see Maheshwari, et al., ‘Pilot study on monitoring ulcer healing with diffuse optical imaging in a patient cohort affected by peripheral arterial disease (PAD)’, 2022)(see A. Marone, et al., ‘Dynamic vascular optical spectroscopy for monitoring peripheral arterial disease patients undergoing a surgical intervention’, 2022)(see A. Marone et al., ‘Using dynamic vascular optical spectroscopy to evaluate peripheral arterial disease (PAD) in patients who undergo a vascular intervention’, 2019)(see H. K. Kim, et al., ‘PDE-constrained multispectral imaging of tissue chromophores with the equation of radiative transfer’, 2010)

### Prototype Development

This technology and the accompanying software have been adapted for developing our first prototype of CaroSense and a PoC study was conducted with 20 healthy subjects. Through the analysis of this preliminary data, the second iteration of CaroSense has been developed with advanced hardware and software changes to optimize the system for improved carotid artery monitoring. These changes include design modifications for user comfortability, enhancing the depth resolution of the system, and enabling 3D image reconstructions of the vasculature in the neck.

The existing probes conform to the neck but are still printed on rigid PCBs and encased in a bulky protective covering. To maximize user comfort, flexible PCBs were designed that more easily fit different neck sizes and better adhere to the tissue. Additionally, the current CaroSense prototype will transition to a wireless and/or handheld design for portability and commercial applications.

Two different interfaces were designed that are optimized for two potential use cases. For the hospital setting, the GUI was changed so that it can be easily integrated with existing hospital alert systems. For the home setting, the interface was simplified and designed for use on a browser or as a mobile application to make it more accessible for users.

The CaroSense device from FIG. 7 was used to monitor the left and right carotid arteries of 20 healthy volunteers. An ultrasound technician was used to identify the locations of the left and right carotid arteries via Doppler ultrasound to place the probes accordingly for acquisition. The data acquired from the carotid arteries was compared to oxygen saturation data acquired from the left and right radial arteries to validate the use of CaroSense for carotid artery monitoring. (see N. Maheshwari et al. ‘Preliminary study using wearable near-infrared spectroscopy for continuous monitoring of hemodynamics through the carotid artery’, 2025) (see N. Maheshwari et al. ‘Preliminary study to validate the use of dynamic optical spectroscopy to monitor oxygen saturation changes in the carotid artery’, 2025)(see L. Sharma et al. ‘Design of a flexible dynamic optical spectroscopic system for monitoring blood oxygenation status in the carotid artery’, 2024)

In addition to testing the functionality of the system, user tests were run to determine changes that should be made to the CaroSense pre-commercialization. This testing was done both with physicians during the preliminary study and with patients who may consider using the system for at-home applications. Furthermore, pre-clinical validation was performed on the developed CaroRhythm device to sense and activate the baroreceptor signaling pathway on micro-physiological systems and ex-vivo models that mimic the carotid artery system.

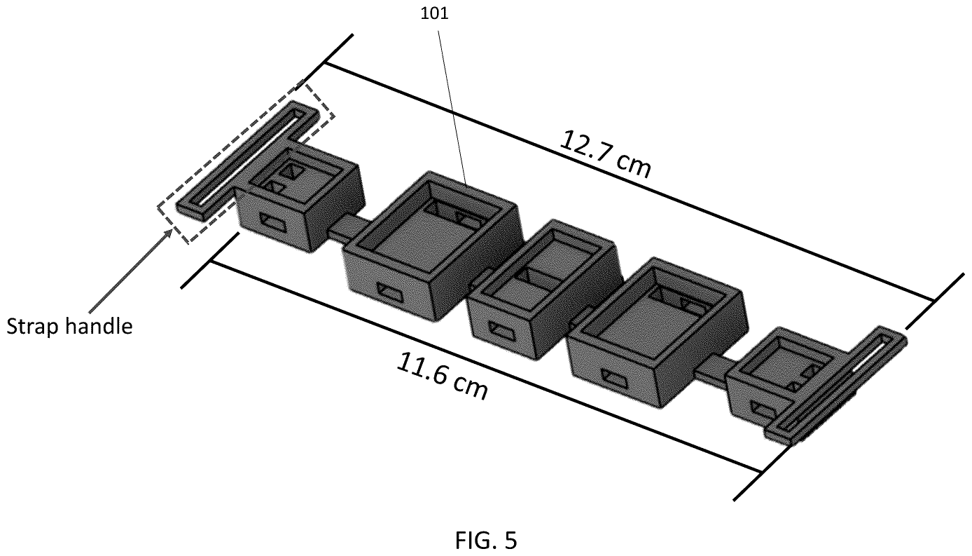

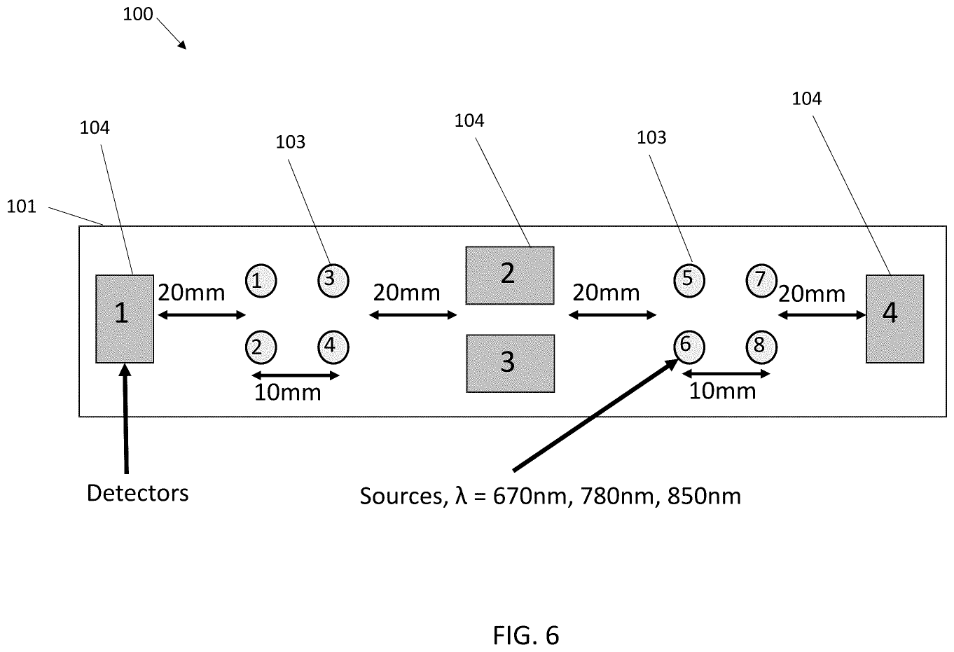

A second iteration prototype device was created (FIGS. 4-6) to further test the validity of the system and enhance its functionality. The device included a 3D printed housing. The patch had a full length of 12.7 cm including handles for neck strap, 11.6 cm considering only hardware storage portion (see FIGS. 4-5). A fabric neck strap was looped through patch handles and adheres each patch to the left and right sides of a subject's neck, respectively. Wires fed through designated slots in the back of the 3D printed housing and connected to a rectangular communications module (FIG. 4) which hung loosely around the subject's neck. The communication module had an opening for the cables that connect to the data acquisition hardware. The new prototype allows for greater flexibility/curvature and adherence to neck.

One patch had 4 detectors and 8 sources (FIGS. 4-6) thus providing 32 source-detector pairs per patch. The sourcing of sources and detectors remained the same as the prior prototype (FIG. 7).

Detector 1 was located approximately 23 mm from sources 1 and 2, 33 mm from sources 3 and 4, 79 mm from sources 5 and 6, and 89 mm from sources 7 and 8.

Detectors 2 and 3 were located approximately 33 mm from sources 1, 2, 7, and 8, and 23 mm from sources 3, 4, 5, and 6,

Detector 4 followed an inverse geometry of Detector 1 and was located approximately 23 mm from sources 7 and 8, 33 mm from sources 5 and 6, 79 mm from sources 3 and 4, and 89 mm from sources 1 and 2.

Each of the eight sources emitted a wavelength of 670 nm, 780 nm, and/or 850 nm. The new acquisition time (frame rate) for simultaneously acquiring data from both the left and right carotid was 0.93 Hz (64 signals per frame).

Furthermore, the software was updated. 3D image reconstruction of both sides of the neck was possible given the increase in source-detector distance and number of signals acquired. Reconstruction was performed using a generalized equality constrained inverse solver (see U.S. Ser. No. 16/211,693, incorporated herein by reference in its entirety). Different methods were used to solve the inverse problem, including an integro-differential diffusion equation and a third-order spherical harmonics algorithm. Geometry-specific mesh generation was done for each subject's neck measurements and patch placement, yielding optimized reconstructions. The CaroSense device will transition to a geometry-independent algorithm to make the device easier to use, less dependent on individual anatomy, and less disruptive to the current workflow

### Experimental Results

It was found through the PoC testing with the first CaroSense prototype that there are no statistically significant differences in changes in oxy, deoxy, or total hemoglobin concentration in response to a 30 second breath hold between the left radial and left carotid arteries (student's t-test, p-value based on Bonferroni correction) which was expected for healthy subjects. Further, there was no statistically significant difference in change in deoxyhemoglobin concentration in response to a 30 second breath hold between the right radial and right carotid arteries (student's t-test, p-value based on Bonferroni correction) which is also expected for healthy subjects.

There were statistically significant differences in oxyhemoglobin concentration change and total hemoglobin concentration change between the right radial and right carotid arteries (student's t-test, p-value based on Bonferroni correction) which was not expected for healthy subjects but was likely due to noise/instrumentation error in patch 3 (the right radial).

There were no statistically significant differences in changes in oxy, deoxy, or total hemoglobin concentration in response to a 30 second breath hold between the left and right carotid arteries which was expected for healthy subjects.

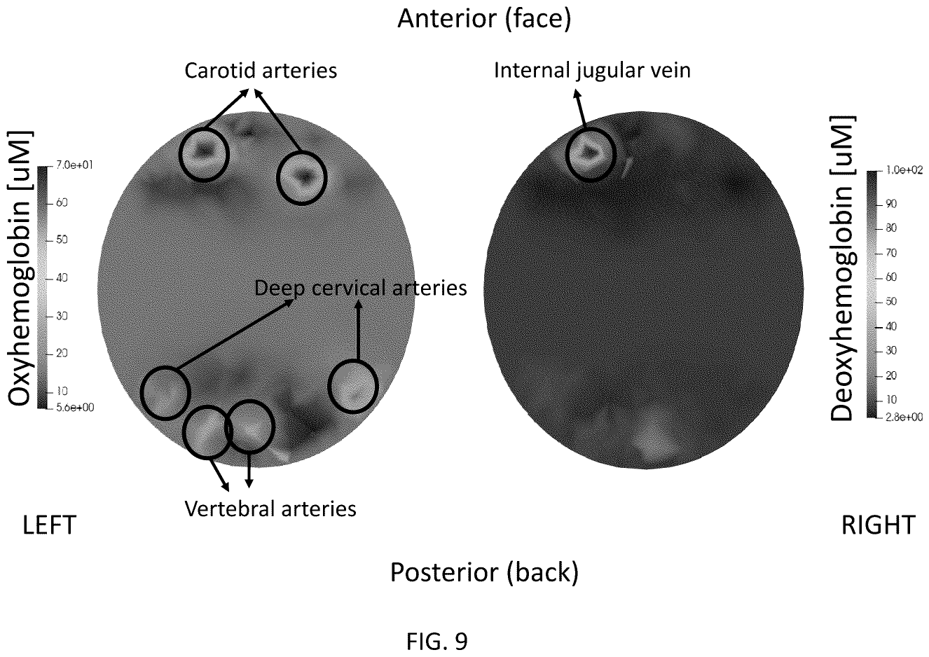

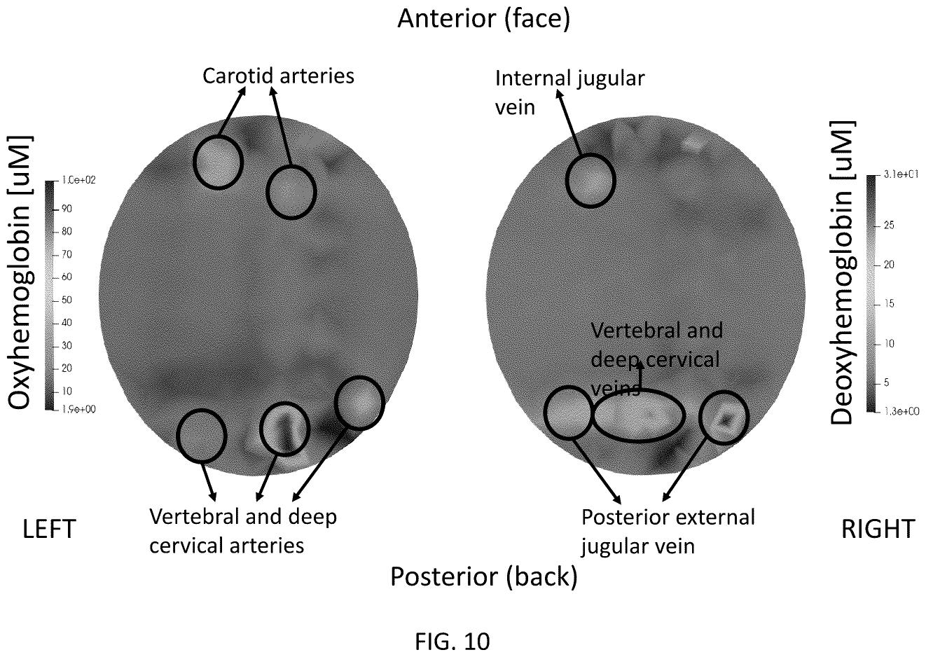

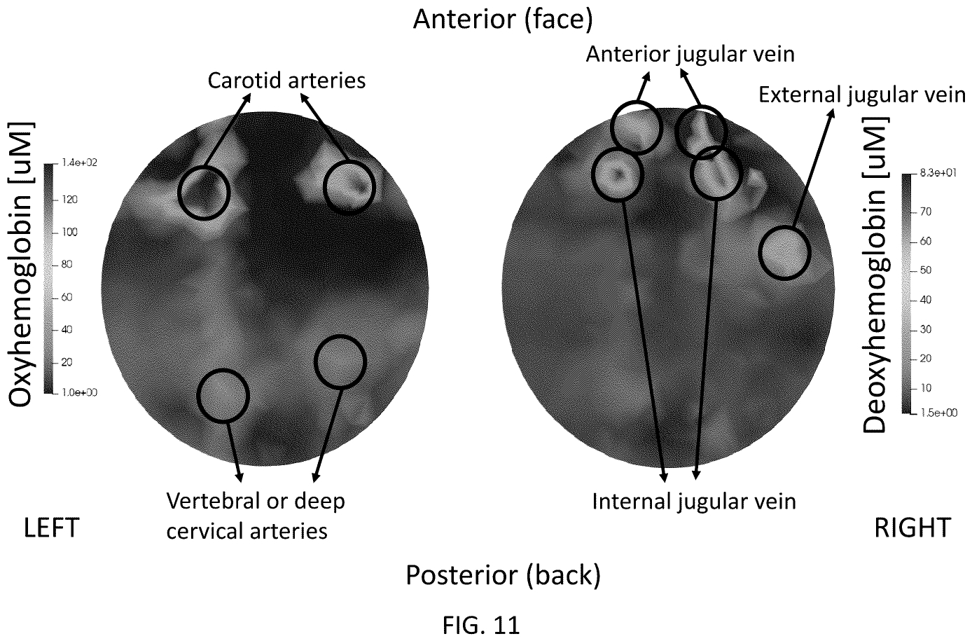

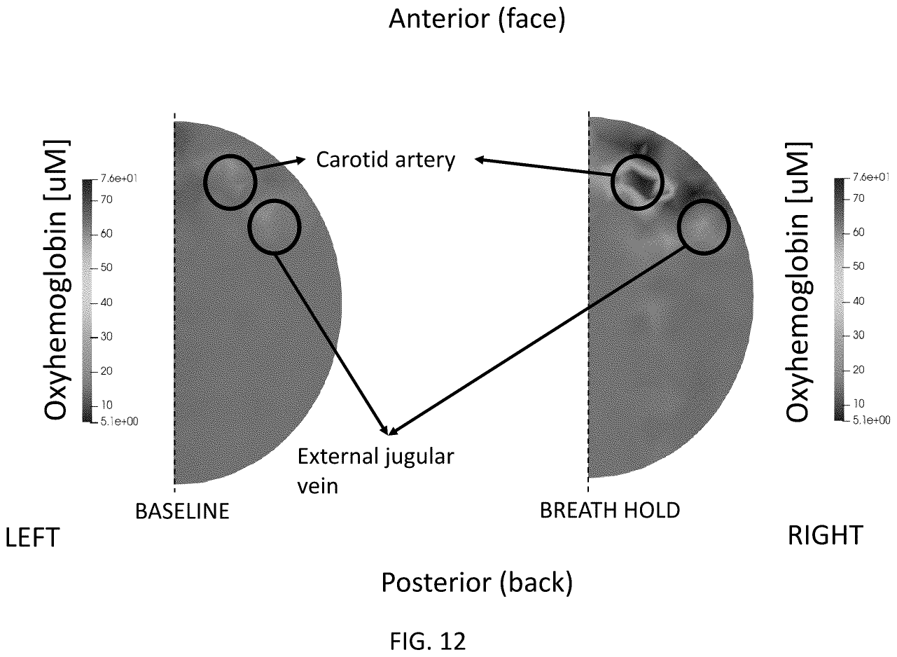

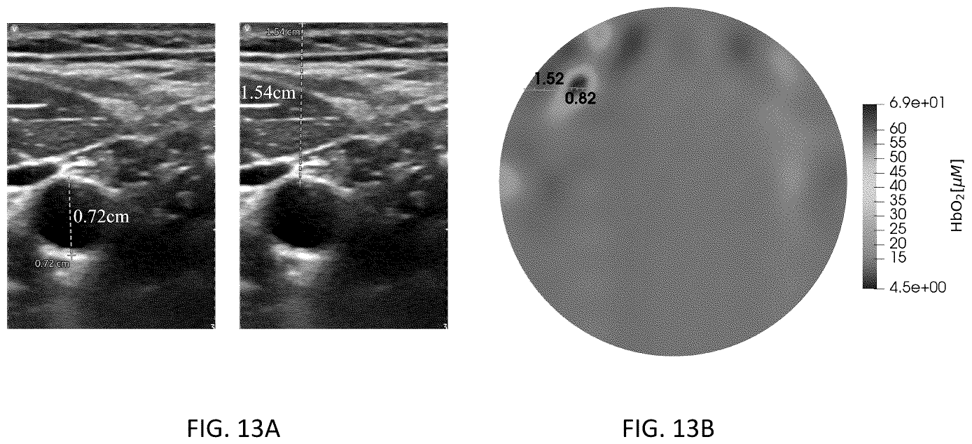

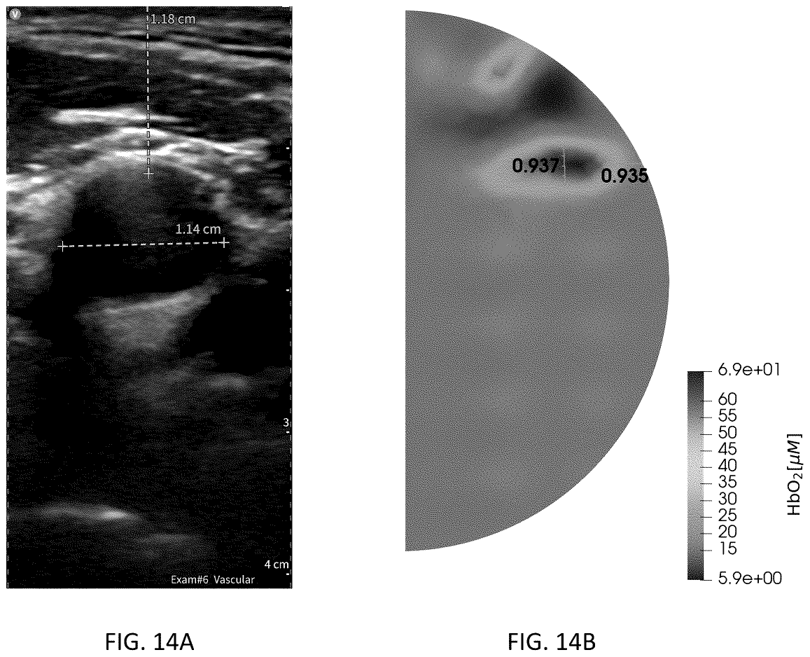

3D image reconstructions in 4 healthy subjects using the device from FIG. 4 match what we expect to see (FIGS. 9-11,13) at peak of breath hold (based on total hemoglobin and/or oxygenated and deoxygenated hemoglobin concentration reconstruction). Dynamic data processing was also done (FIG. 12) and shows the difference in oxygenated blood flow during baseline versus at the peak of breath hold in the same subject. FIGS. 9-11 and FIGS. 13-14 show changes with respect to baseline (defined as the average of the first 20 frames of data, or approximately the first 19 seconds of data collection) and the reconstructed images are axial cross sections which correspond to the center (relative to the height) of each patch. Anatomic assumptions were validated with carotid Doppler ultrasound for the subjects corresponding to FIGS. 10, 12, & 13 and the images reconstructed with CaroSense data were within 2 mm spatial resolution (FIGS. 13-14) of the ultrasound measurements.

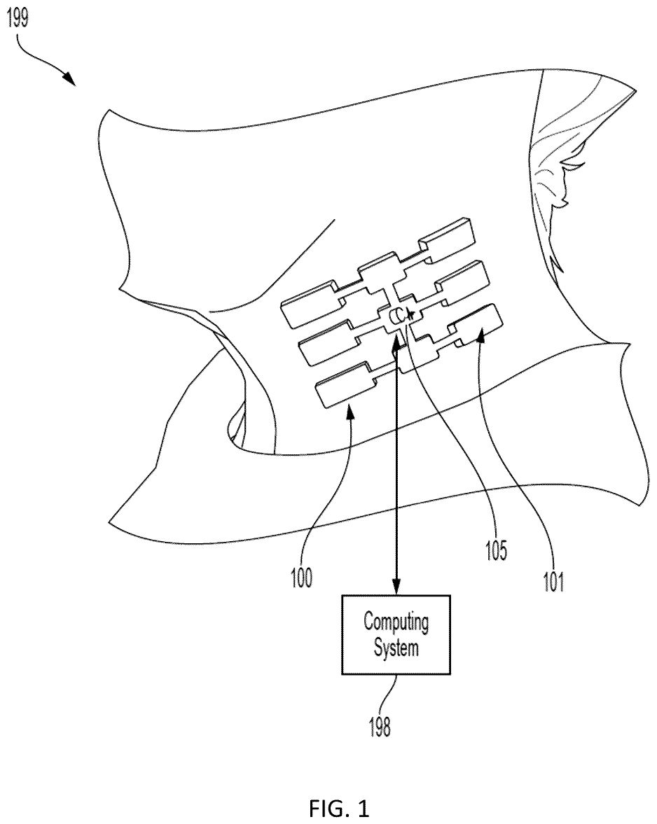

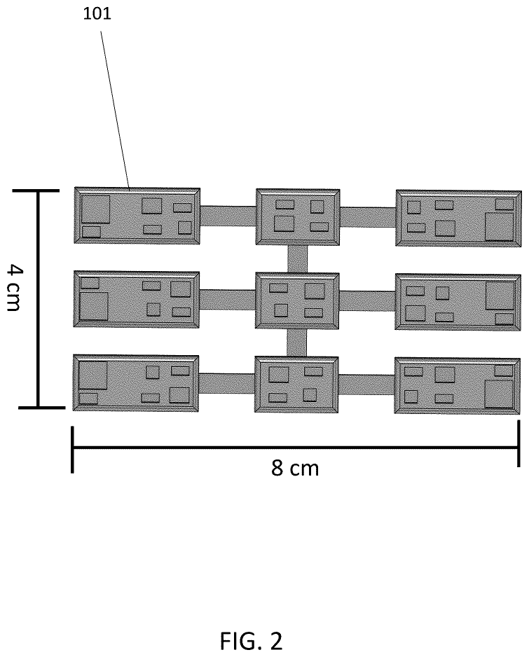

Another prototype device was created (FIGS. 1-3) to add the ability to track and distinguish plaque in the carotid arteries to the existing 3D imaging functionality. Short-wave infrared spectral wavelengths in the 1200-1500 nm range were added to optimize the absorption coefficients of different types of lipids and cholesterol. The patch, including housing, measures 4 cm by 8 cm (FIG. 2).

One patch had 12 detectors and 16 sources (FIG. 2), where 6 silicon detectors were specifically for capturing light in the near-infrared range, 6 InGaAs detectors were specifically for capturing light in the short-wave infrared range, 8 light sources have wavelengths in the near-infrared range, and 8 light sources have wavelengths in the short-wave infrared range. Thus there were 48 source-detector pairs for monitoring hemodynamics and 48 source-detector pairs for monitoring embolic activity.

The sources were light-emitting diodes. The eight sources in the near-infrared wavelength range emit at a wavelength of 780 nm and/or 850 nm (sourced from Luminus devices: MP-2835-1100-FR and Vishay Semiconductor: VSMY2850GX01, respectively). The eight sources in the short-wave infrared wavelength range emit at a wavelength of 1200 nm and/or 1460 nm (sourced from Marktech Optoelectronics: MTSM 0012-844-IR, MTSM 6014-844-IR). The light-emitting diodes for the 780 nm, 850 nm, 1200 nm, and 1460 nm operate at 62 mW, 55 mW, 12 mW, and 6 mW radiant power, respectively.

The six silicon detectors for the near-infrared range detect light in the 400-1100 nm range (sourced from ams OSRAM: SFH 2703). The six InGaAs detectors for the short-wave infrared range detect light in the 600-1750 nm range (sourced from Marktech Optoelectronics: MTSM1346SMF2-150).

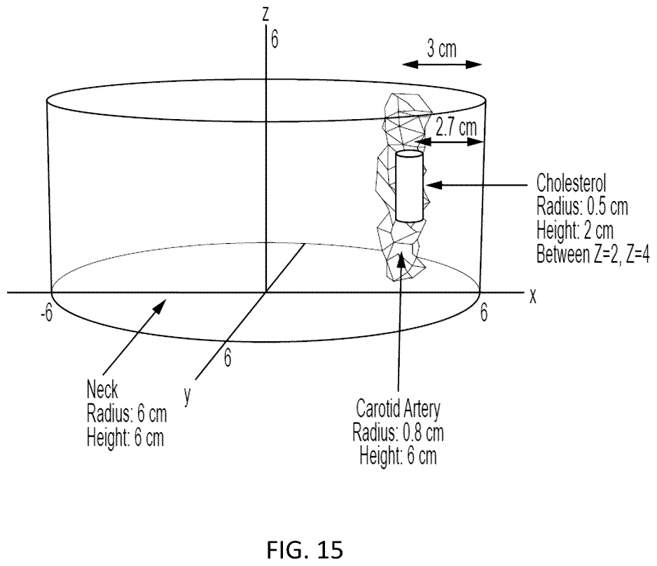

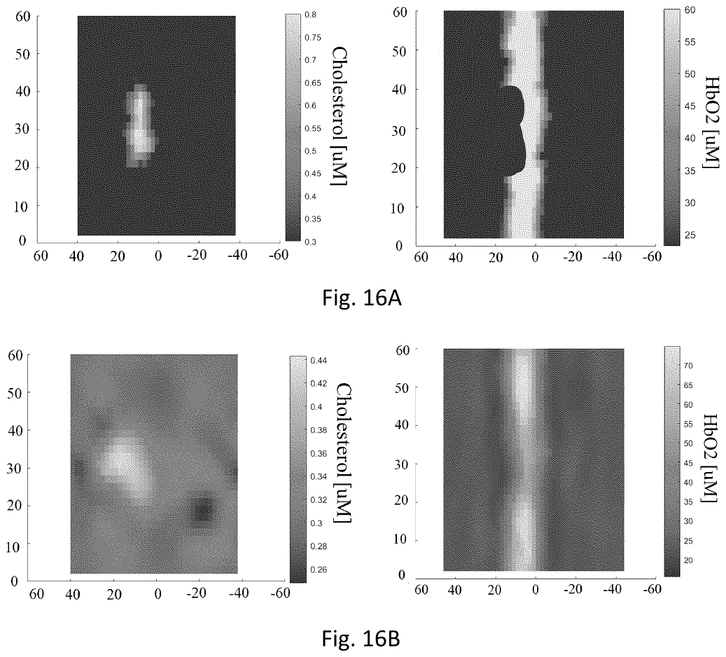

Using the new prototype, software simulations indicate it is possible to detect plaque within the carotid artery. A simulated cylinder of plaque with radius of 0.5 cm and height of 2 cm located in a simulated carotid artery with radius of 0.8 cm and height of 6 cm (FIG. 15) was generated. Simulations show that the device of FIGS. 1-3 can resolve the height and location of the plaque (FIG. 16). The software employed the same generalized equality constrained inverse solver used prior (using the integro-differential diffusion equation). The optimized software provided a spatial resolution of less than 5 mm.

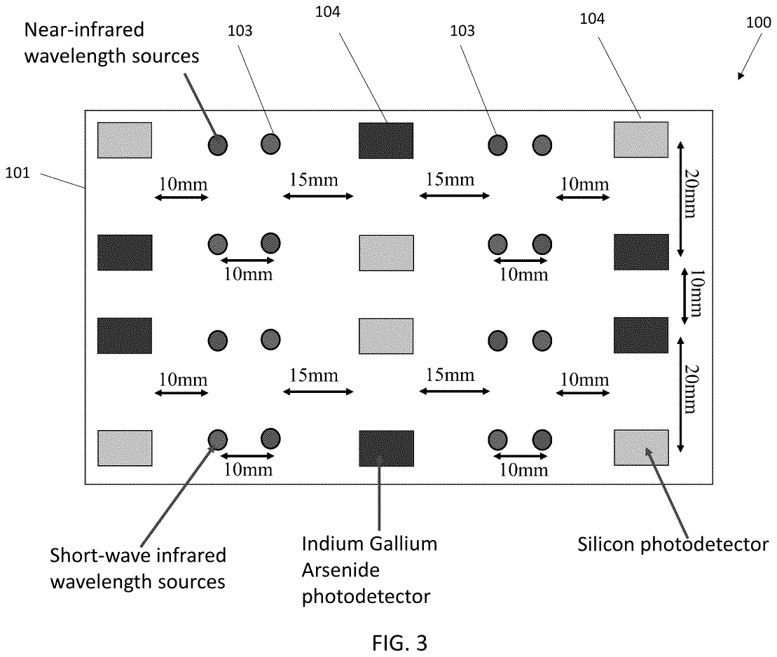

In another embodiment (FIG. 3), the patch had 12 detectors and 36 sources, where 6 silicone detectors were specifically for capture light in the near-infrared range, 6 InGaAs detectors were specifically for capturing light in the short-wave infrared range, 18 light sources have wavelengths in the near-infrared range, and 18 light sources have wavelengths in the short-wave infrared range. Thus, this embodiment has 108 source-detector pairs for monitoring hemodynamics and 108 source-detector pairs for monitoring embolic activity. The light sources and photodetectors are sourced from the same place as the embodiment in FIG. 2 detailed above.

### Software Implementation

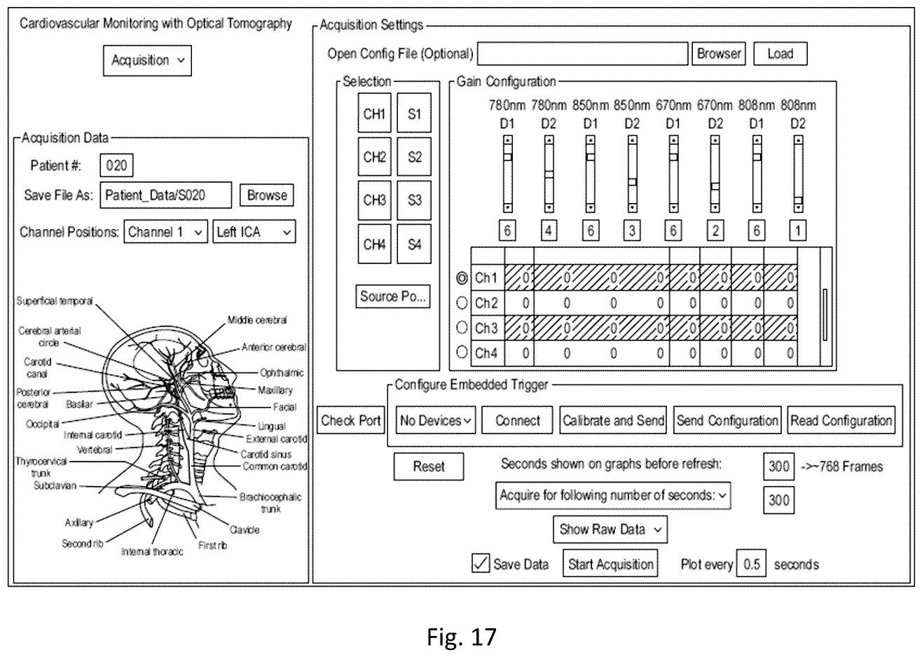

A graphical user interface (GUI) has been developed in MATLAB as part of the CaroSense prototype. The user controls when the CaroSense patch(es) starts collecting data, the duration of the data acquisition period, and when to stop acquiring data via the GUI (FIG. 17). For the first two iterations of CaroSense prototypes, the photodetectors (FIGS. 4-7) continuously acquire the incremental changes in reflected light due to absorption, and send the reflected light intensity information to the GUI using an SPI protocol. This information is displayed in the GUI and updated in real-time for all source-detector pairs from all patches (up to 64 signals can be tracked in the GUI, as there are 32 source-detector pairs per the two patches in the prototype corresponding to FIGS. 4-6, one patch for each carotid artery).

Intensity data can be converted into the corresponding oxygenated and deoxygenated hemoglobin concentrations over time. This is done via a diffusion-theory-based PDE-constrained multispectral reconstruction algorithm. (see H. K. Kim, et al., ‘PDE-constrained multispectral imaging of tissue chromophores with the equation of radiative transfer’, 2010) The oxygen saturation over time (StO2) can also be plotted, which allows the user to track dynamic changes in StO2 in real time. The user can also be alerted when large changes in StO2 are detected.

Users can save data via the GUI after they stop acquiring data. Currently, the data (raw signals and hemodynamic information) are uploaded to a database.

### Computing Environment

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

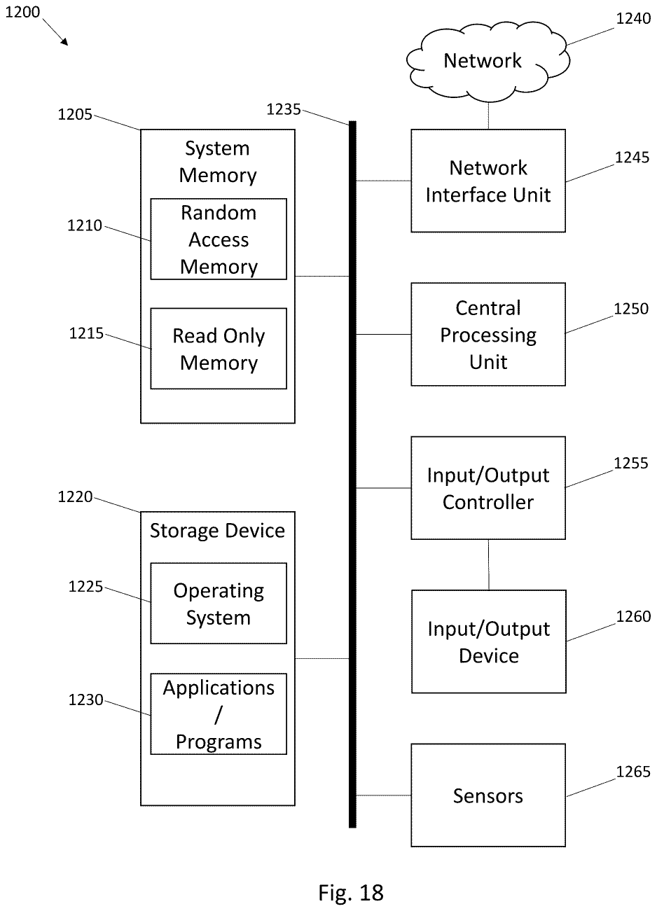

FIG. 18 and the following discussion are intended to provide a brief, general description of a suitable computing enviro nment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing enviro nments where tasks are performed by remote processing devices that are linked through a communications network. In a distributed computing enviro nment, program modules may be located in both local and remote memory storage devices.

FIG. 18 depicts an illustrative computer architecture for a computer 1200 for practicing the various embodiments of the invention. The computer architecture shown in FIG. 18 illustrates a conventional personal computer, including a central processing unit 1250 (“CPU”), a system memory 1205, including a random-access memory 1210 (“RAM”) and a read-only memory (“ROM”) 1215, and a system bus 1235 that couples the system memory 1205 to the CPU 1250. A basic input/output system containing the basic routines that help to transfer information between elements within the computer, such as during startup, is stored in the ROM 1215. The computer 1200 further includes a storage device 1220 for storing an operating system 1225, application/program 1230, and data.

The storage device 1220 is connected to the CPU 1250 through a storage controller (not shown) connected to the bus 1235. The storage device 1220 and its associated computer-readable media, provide non-volatile storage for the computer 1200. Although the description of computer-readable media contained herein refers to a storage device, such as a hard disk or CD-ROM drive, it should be appreciated by those skilled in the art that computer-readable media can be any available media that can be accessed by the computer 1200.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 1200 may operate in a networked enviro nment using logical connections to remote computers through a network 1240, such as TCP/IP network such as the Internet or an intranet. The computer 1200 may connect to the network 1240 through a network interface unit 1245 connected to the bus 1235. It should be appreciated that the network interface unit 1245 may also be utilized to connect to other types of networks and remote computer systems.

The computer 1200 may also include an input/output controller 1255 for receiving and processing input from a number of input/output devices 1260, including a keyboard, a mouse, a touchscreen, a camera, a microphone, a controller, a joystick, or other type of input device. Similarly, the input/output controller 1255 may provide output to a display screen, a printer, a speaker, or other type of output device. The computer 1200 can connect to the input/output device 1260 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 1220 and RAM 1210 of the computer 1200, including an operating system 1225 suitable for controlling the operation of a networked computer. The storage device 1220 and RAM 1210 may also store one or more applications/programs 1230. In particular, the storage device 1220 and RAM 1210 may store an application/program 1230 for providing a variety of functionalities to a user. For instance, the application/program 1230 may comprise many types of programs such as a word processing application, a spreadsheet application, a desktop publishing application, a database application, a gaming application, internet browsing application, electronic mail application, messaging application, and the like. According to an embodiment of the present invention, the application/program 1230 comprises a multiple functionality software application for providing word processing functionality, slide presentation functionality, spreadsheet functionality, database functionality and the like.

The computer 1200 in some embodiments can include a variety of sensors 1265 for monitoring the enviro nment surrounding and the enviro nment internal to the computer 1200. These sensors 1265 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

### References

The following publications are each hereby incorporated herein by reference in their entirety:
1). Maheshwari N, Marone A, Sharma L, Kim S, Favate A, Hielscher A H. Preliminary study using wearable near-infrared spectroscopy for continuous monitoring of hemodynamics through the carotid artery. Biosensors. 2025 Aug. 20; 15(8). Available from: https://doi.org/10.3390/bios15080549 DOI:10.3390/bios15080549
2). Maheshwari N, Sharma L, Kim SHK, Marone A, Favate A, Hielscher A H. Preliminary study to validate the use of dynamic optical spectroscopy to monitor oxygen saturation changes in the carotid artery. Optical Diagnostics and Sensing XXV: Toward Point-of-Care Diagnostics. SPIE Photonics West BiOS; 2025; San Francisco, CA, United States. SPIE; c2025. Available from: https://doi.org/10.1117/12.3043716 DOI:10.1117/12.3043716
3). Sharma L, Maheshwari N, Marone A, Kim S H K, Favate A, Hielscher A H. Design of a flexible dynamic optical spectroscopic system for monitoring blood oxygenation status in the carotid artery. Optical Diagnostics and Sensing XXIV: Toward Point-of-Care Diagnostics. SPIE Photonics West BiOS; 2024 January; San Franscisco, CA, United States. SPIE; c2024. Available from: https:/www.spiedigitallibrary.org/conference-proceedings-of-spie/12850/128500O/ Design-of-a-flexible-dynamic-optical-spectroscopic-system-for-monitoring/ 10.1117/12.3003762.short DOI:10.1117/12.3003762
4). M. A. Khalil et al., ‘Detection of Peripheral Arterial Disease Within the Foot Using Vascular Optical Tomographic Imaging: A Clinical Pilot Study’, Eur. J. Vasc. Endovasc. Surg. Off. J. Eur. Soc. Vasc. Surg. , 49(1), 83-89 (2015), doi:10.1016/j.ejvs.2014.10.010.
5). N. Maheshwari et al. ‘Postintervention monitoring of peripheral arterial disease wound healing using dynamic vascular optical spectroscopy,’ J. Biomed. Opt. 27(12), 125002 (2022), doi:10.1117/1.JBO.27.12.125002.
6). N. Maheshwari, et al., ‘Pilot study on monitoring ulcer healing with diffuse optical imaging in a patient cohort affected by peripheral arterial disease (PAD)’, in Optical Diagnostics and Sensing XXII: Toward Point-of-Care Diagnostics, San Francisco, United States, March 2022, p. 18. doi:10.1117/12.2610373.
7). A. Marone, et al., ‘Dynamic vascular optical spectroscopy for monitoring peripheral arterial disease patients undergoing a surgical intervention’, Front. Photonics, 3, 938144, (2022), doi:10.3389/fphot.2022.938144.
8). A. Marone et al., ‘Using dynamic vascular optical spectroscopy to evaluate peripheral arterial disease (PAD) in patients who undergo a vascular intervention’, in Optical Tomography and Spectroscopy of Tissue XIII, San Francisco, United States, March 2019, p. 14. doi:10.1117/12.2509116.
9). H. K. Kim, et al., ‘PDE-constrained multispectral imaging of tissue chromophores with the equation of radiative transfer’, Biomed. Opt. Express, 1(3), 812 (2010), doi:10.1364/BOE.1.000812.
10). Huang, Li-Da. “Brighten the future: Photobiomodulation and optogenetics.” Focus 20.1 (2022):36-44.
11). Cheng, M. Y., Wang, E. H., & Steinberg, G. K. (2014). Optogenetic approaches to study stroke recovery. ACS chemical neuroscience, 5(12), 1144-1145.
12). Forbes, Jessica, and Ritesh G. Menezes. “Anatomy, head and neck, carotid bodies.” (2020).
13). Biscoe, T. J. “Carotid body: structure and function.” Physiological Reviews 51.3 (1971):437-495.
14). Eyzaguirre, C., and P. Zapata. “Perspectives in carotid body research.” Journal of Applied Physiology 57.4 (1984):931-957.
15). Shirahata, M., et al. “Is the carotid body a metabolic monitor?” Arterial Chemoreceptors in Physiology and Pathophysiology (2015):153-159.
16). U.S. patent application Ser. No. 16/211,693, titled “Tomographic Imaging Methods, Devices, and Systems”, filed Dec. 6, 2018.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. An optical spectroscopic device for cardiovascular monitoring, comprising:
a housing;
an attachment mechanism for attaching the housing proximate to a target tissue location;
a plurality of light sources positioned on the housing, each configured to emit light at one of a plurality of wavelengths;
a plurality of photodetectors proximate to the light sources and positioned on the housing, configured to measure the light emitted by the light sources that transmits through or reflects off the target tissue location;
a transducer configured to convert voltage levels measured at the plurality of photodetectors to digital measurement data; and
a communications module electrically connected to the transducer, configured to transmit the digital measurement data to a computing system configured to calculate a property of the blood within the target tissue location based on the digital measurement data.

2. The device of claim 1, wherein the light sources comprise light-emitting diodes.

3. The device of claim 1, wherein the photodetectors comprise silicon and indium gallium arsenide photodiodes.

4. The device of claim 1, wherein the light sources are positioned in a columnar configuration.

5. The device of claim 1, wherein the attachment mechanism comprises biocompatible adhesive.

6. The device of claim 1, wherein each of the photodetectors is positioned 10 mm to 115 mm from at least one of the light sources.

7. The device of claim 1, wherein the housing is flexible.

8. The device of claim 1, wherein the plurality of light sources comprises 16 light sources and the plurality of photodetectors comprises 12 photodetectors.

9. The device of claim 1, wherein the device is configured for placement proximate to a carotid artery.

10. The device of claim 1, wherein all of the wavelengths emitted by the light sources are in the range of 650 nm to 1500 nm.

11. The device of claim 1, wherein the plurality of light sources are positioned in first and second groups, with a first group of four of the plurality of photodetectors positioned between the first and second groups of light sources, a second group of four of the plurality of the photodetectors positioned on a side of the first group of light sources opposite the first group of photodetectors, and a third group of four of the of the plurality of the photodetectors positioned on a side of the second group of light sources opposite the first and second groups of photodetectors.

12. The device of claim 1, wherein the device is configured to remotely monitor for strokes.

13. The device of claim 1, wherein the device is configured for outpatient monitoring of the carotid artery.

14. The device of claim 1, wherein the device is configured to determine blood pH.

15. The device of claim 1, wherein the device is non-invasive.

16. The device of claim 1, wherein the device is configured to measure plaque composition.

17. The device of claim 1, wherein the device is configured to track the movement of plaque through the carotid artery.

18. The device of claim 1, wherein the device is configured to provide 3D images of the vasculature in the neck.

19. An optical spectroscopic system for cardiovascular monitoring, comprising:
the optical spectroscopic device of claim 1; and
where the computing system communicatively connected to the optical spectroscopic device comprises a processor and a non-transitory computer-readable medium with instructions stored thereon, which when executed by the processor, perform steps comprising:
applying light of several wavelengths to the target tissue location;
receiving the digital measurement data transmitted by the optical spectroscopic device; and
calculating a property of at least one of the blood, the plaque, and the carotid artery within the target tissue location based on the digital measurement data.

20. A cardiovascular monitoring method, comprising:
providing the optical spectroscopic device of claim 1;
applying a plurality of wavelengths of light to the target tissue location with the plurality of light sources;
measuring transmission or reflection of the target tissue location with the plurality of photodetectors; and
calculating a property at least one of the blood, the plaque, and the carotid artery within the target tissue location.

21. The method of claim 20, wherein the calculated property comprises at least one of blood pH, plaque composition, plaque location, and blood oxygenation.
